(19) United States
(12) Patent Application Publication
(10) Pub. No.: US 2017/0014105 A1
(43) Pub. Date: Jan. 19, 2017

(54) ULTRASONIC DIAGNOSTIC DEVICE

(71) Applicant: HITACHI, LTD., Tokyo (JP)

(72) Inventor: Tomoaki CHONO, Tokyo (JP)

(73) Assignee: Hitachi, Ltd., Tokyo (JP)

(21) Appl. No.: 15/121,129

(22) PCT Filed: Nov. 5, 2014
(86) PCT No.: PCT/JP2014/079344

§ 371 Date: Aug. 24, 2016

(30) Foreign Application Priority Data
Feb. 24, 2014 (JP) 2014-033296

(51) Int. Cl. A61B 8/08; A61B 8/00; A61B 8/06

(52) U.S. Cl. CPC A61B 8/488; A61B 8/06; A61B 8/5207; A61B 8/461; A61B 8/4483

(57) ABSTRACT

A tomographic image forming unit forms a tomographic image of a target tissue on the basis of received signals which are obtained by sending and receiving ultrasound waves to and from the target tissue. A tomographic image analysis unit analyzes a formed tomographic image with image processing techniques and extracts a reference portion on the target tissue. On the basis of the extracted reference portion, a Doppler measurement position specification unit specifies a Doppler measurement position. A Doppler waveform forming unit performs Doppler measurement at the specified Doppler measurement position and forms a Doppler waveform. The formed tomographic image, a cursor which indicates the specified Doppler measurement position, and the formed Doppler waveform are displayed on a display unit. When specifying the Doppler measurement position, a color Doppler image may also be referred to.

### TECHNICAL FIELD

The present invention relates to an ultrasonic diagnostic device, and more particularly to a technique of automatically setting a measurement position for Doppler measurement.

### BACKGROUND

Ultrasonic diagnostic devices transmit and receive ultrasound waves to and from an examinee, and, based on a received signal thus obtained, form an ultrasound image. The ultrasonic diagnostic devices have a Doppler measurement function for detecting the moving direction and the moving speed of a target subject using a Doppler effect. The Doppler measurement function is used for measuring a blood flow velocity, for example.

Several Doppler measurement methods are known, including, for example, a method called color Doppler, for performing Doppler measurement in a wide range to obtain a color Doppler image that indicates distribution of the velocity of blood flow within the range, a method called continuous wave Doppler, for performing Doppler measurement using continuous waves over a wide range on an ultrasound beam, and a method called pulsed Doppler for performing Doppler measurement using pulse waves in a local area on an ultrasound beam. Any of these methods requires appropriate setting of a measurement range or measurement position. A user moves area frames, lines, and cursors on the screen with a track ball or the like provided on an operation panel to thereby specify the measurement range or the measurement position.

For measuring the left ventricular inflow blood velocity of the heart, for example, the user sets the measurement position (cursor) for pulsed Doppler on a blood flow portion near the mitral valve. For measuring the right ventricle inflow blood velocity, the measurement position is set on a blood flow portion near the tricuspid valve for performing the Doppler measurement. For measuring the left ventricular outflow blood velocity, the measurement position is set on a blood flow portion near the main artery for performing the Doppler measurement. To measure the blood flow velocity accurately at each position, it is important to set the Doppler measurement position accurately. Skilled specification operations are therefore required for users, who must perform such a complicated operation each time the Doppler measurement is performed. To address this deficiency, a technique for automatically setting the measurement position for the Doppler measurement is desired.

Patent Document 1 suggests a method for automating an operation of specifying the direction and depth of a Doppler mode to increase the accuracy in setting a cursor (Doppler measurement position). The apparatus described in Patent Document 1 detects a position where the velocity of blood flow is the maximum in the distribution data of the velocity of blood flow indicated by color Doppler and sets a measurement position of the continuous wave Doppler and pulsed Doppler at this detected position. The apparatus also extracts a point where the velocity of the blood flow is the maximum from color image data of a plurality of frames obtained within one heartbeat period, and sets this point as a Doppler measurement position.

### CITATION LIST

### Patent Literature

Patent Document 1: JP2002-306485 A

### SUMMARY

### Technical Problem

In the invention described in Patent Document 1, the measurement position for the Doppler measurement is set based only on the blood flow velocity. However, because the blood flow velocity usually changes drastically, and also because of the aliasing effects, there is a possibility that the accuracy in the Doppler measurement position is questionable. Alternatively, when the position where the blood flow is the maximum is located at a position other than the Doppler measurement position which is desired by the user, the measurement position is be set at an undesirable position. As such, there is also a possibility that the Doppler measurement position is not always set to a position desired by the user.

The present invention is aimed at providing an ultrasonic diagnostic device that automatically sets a Doppler measurement position with high accuracy.

### Solution to Problem

In accordance with an aspect of the invention, an ultrasonic diagnostic device includes a tomographic image forming unit configured to form, based on a received signal obtained by transmitting and receiving an ultrasound wave to and from a beam scanning area including a target tissue in which blood flows, a tomographic image of the target tissue, a tomographic image analysis unit configured to analyze the tomographic image to extract a reference portion in the target tissue, a position specification unit configured to specify a Doppler measurement position within the target tissue based on the reference portion, and a Doppler waveform forming unit configured to form a Doppler waveform showing a movement of blood flow in the Doppler measurement position, based on a received signal obtained by transmitting and receiving an ultrasound wave to the Doppler measurement position. Preferably, the target tissue is a heart, and the reference portion is an annulus portion of the heart or a contour of a heart cavity.

The above structure allows extraction, from a tomographic image formed based on a received signal obtained by transmission and reception of ultrasound waves, of a reference portion as a specific tissue image or a specific tissue position in a target tissue included in the tomographic image. The reference portion is extracted in order to specify a Doppler measurement position. The reference portion may be extracted in accordance with the purpose, subject, and the like of the Doppler measurement. Once the reference portion is extracted, the Doppler measurement position is specified within the target tissue based on the reference portion. The reference portion and the Doppler measurement position may be different positions, and, based on a relation expression indicating a positional relationship between the reference portion and the Doppler measurement position, the Doppler measurement position may be specified from the reference portion. With the Doppler measurement (continuous wave Doppler measurement, pulsed Doppler measurement) performed in the specified Doppler measurement position, a Doppler waveform indicating the motion of blood flow in the Doppler measurement position is generated.

As the Doppler measurement is aimed at measuring blood flow, the Doppler measurement position is set to a position where blood flows, that is, a position in a tomographic image where a shape cannot be represented. It is therefore difficult to set the Doppler measurement position directly based on the characteristics concerning the shape of blood flow. There is a predetermined positional relationship between an annulus position (reference portion) which is a base portion of the mitral valve in the heart and a Doppler measurement position which is suitable for measuring the left ventricular inflow blood, for example. Use of such a relationship to specify the Doppler measurement position based on the annulus position which can be easily extracted in a stable manner enables stable setting of the Doppler measurement position with high accuracy.

Preferably, the ultrasonic diagnostic device further includes a blood flow information generation unit configured to generate blood flow information indicating a spatial distribution of velocity of blood flow within the target tissue, based on the received signal, and the position specification unit specifies the Doppler measurement position based on the reference portion and the blood flow information. Preferably, the tomographic image analysis unit defines an analysis range based on the reference portion, and the position specification unit specifies the Doppler measurement position based on a portion in a spatial distribution of the velocity of the blood flow corresponding to a distribution of the velocity within the analysis range. Preferably, the position specification unit specifies the Doppler measurement position based on a position within the analysis range where the velocity of blood flow is the maximum.

Specification of the Doppler measurement position in consideration of not only the reference portion based on the tomographic image of a tissue but also the distribution of the velocity of blood flow within the target tissue allows more accurate setting of the Doppler measurement position to a more appropriate position. In the case of measurement of the left ventricular inflow blood in the heart, for example, a position where the velocity of the blood flow is the maximum is determined as an appropriate Doppler measurement position. In many cases, an appropriate Doppler measurement position is determined in accordance with the velocity of blood flow as described above. Accordingly, specification of the Doppler measurement position in consideration of not only the reference portion but also distribution of the velocity of blood flow allows an increase in the accuracy of the Doppler measurement position.

Preferably, the position specification unit specifies the Doppler measurement position based on the reference portion and the blood flow information in a specific time selected in a pulsation cycle of the target tissue. Further, preferably, the specific time is a time phase in which blood flow in a specific direction is expressed in the Doppler measurement position.

The above structure allows specification of the Doppler measurement position in an appropriate time phase during the pulsation cycle of the target tissue. The distribution of velocity of blood flow in the target tissue may vary depending on the time phase in the pulsation cycle of the target tissue. In the heart, for example, the distribution of the velocity of blood flow within the heart varies significantly between the systole phase and the diastolic phase. Accordingly, in order to measure the left ventricular inflow blood, for example, it is desirable to perform the measurement in a time phase in which the velocity of the left ventricular inflow blood is the maximum. In such a case, it is desirable that the Doppler measurement position is specified to a position where the velocity of the blood flow is the maximum in that time phase. Thus, specification of the Doppler measurement position taking the time phase into consideration enables setting of the Doppler measurement position more accurately.

Preferably, the position specification unit specifies a plurality of Doppler measurement positions. Further, preferably, the position specification unit specifies a plurality of Doppler measurement positions in accordance with flow directions of the blood flow. Further, preferably, the ultrasonic diagnostic device further includes a measurement position selection unit configured to select from among the plurality of Doppler measurement positions a specific Doppler measurement position concerning which a Doppler waveform is to be displayed, and the Doppler waveform forming unit forms a Doppler waveform based on a received signal obtained by transmission and reception of an ultrasound wave to and from the specific Doppler measurement position which is selected.

### Advantageous Effects of Invention

According to the present invention, a Doppler measurement position can be set automatically with high accuracy.

### BRIEF DESCRIPTION OF DRAWINGS

FIG. 1 is a diagram schematically illustrating a structure of an ultrasonic diagnostic device according to an embodiment of the invention.

FIG. 2 is a diagram illustrating an example Doppler measurement position specified based on a reference portion.

FIG. 3 is a diagram illustrating an example Doppler measurement position specified based on distribution of the blood flow velocity.

FIG. 4 is a diagram illustrating an example Doppler measurement position specified based on a reference portion and distribution of the blood flow velocity.

FIG. 5 is a diagram illustrating a plurality of example Doppler measurement positions specified based on a reference portion.

FIG. 6 is a diagram illustrating switching from a pulsed Doppler mode to a continuous wave Doppler mode in a retrograde flow position.

FIG. 7 is a diagram illustrating an example measurement range of color Doppler specified based on a reference portion.

FIG. 8 is a diagram illustrating an example measurement position of tissue Doppler specified based on a reference portion.

FIG. 9 is a flow chart illustrating a flow of operations performed by the ultrasonic diagnostic device according to an embodiment of the invention.

### DESCRIPTION OF EMBODIMENTS

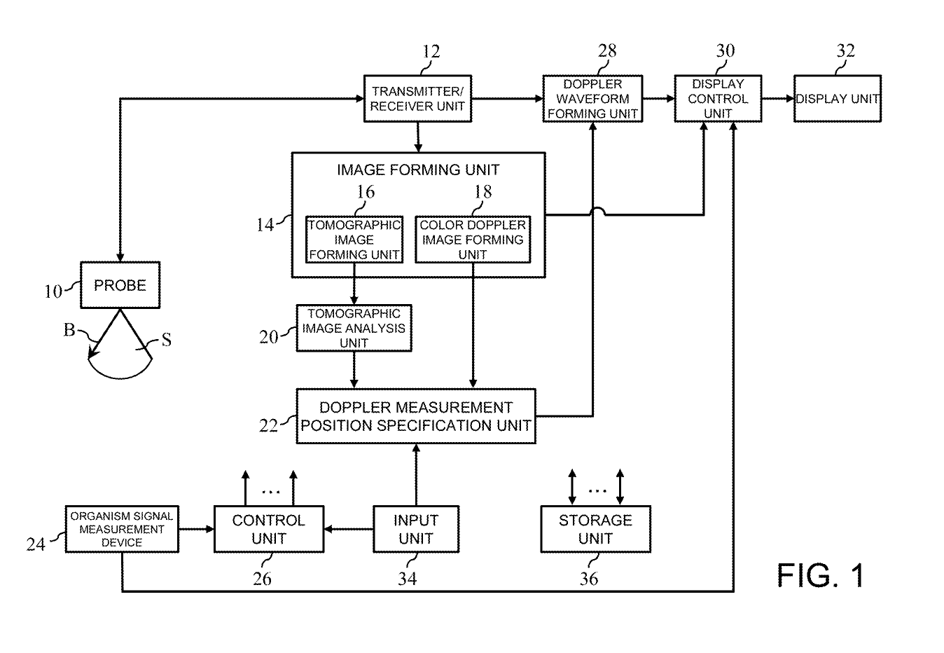

Embodiments of an ultrasonic diagnostic device according to the present invention will be described hereinafter. It should be noted that the present invention is not limited to the following embodiments. FIG. 1 is a diagram schematically illustrating a structure of an ultrasonic diagnostic device according to an embodiment.

A probe 10 is an ultrasound probe which transmits and receives ultrasound waves to and from a target tissue. The target tissue is an organism tissue in which blood flows and, in the present embodiment, is a heart. A blood vessel or other circulatory organ tissues may be the target tissue. The probe 10 includes an array transducer formed of a plurality of transducer elements, and the array transducer forms an ultrasound beam B. Also, a scanning plane S is formed by electronic scanning of the ultrasound beam B. Methods for the electronic scanning include, for example, an electronic sector scanning method and an electronic linear scanning method. The probe 10 may include a 2D array transducer to allow capturing of three-dimensional data. As will be described below, based on a received signal obtained by scanning an ultrasound beam by the probe 10, a tomographic image of a target tissue and a color Doppler image showing a distribution of blood flow within the target tissue are captured. Further, with Doppler observation in a specific orientation (and depth), a Doppler waveform showing a change in the velocity spectrum of the blood flow with time, for example, is formed.

A transmitter/receiver unit 12 transmits a plurality of transmitting signals, which oscillate a plurality of transducer elements of the probe 10, to the probe 10, thereby causing the probe 10 to generate ultrasound waves. The transmitter/receiver unit 12 also performs phase alignment and summation processing with respect to a plurality of received signals obtained from the plurality of transducer elements of the probe 10, thereby forming a received beam, that is, a received signal (beam data) having undergone the phase alignment and summation processing. As such, the transmitter/receiver unit 12 has functions of a transmitting beam former and a received beam former.

An image forming unit 14 forms various images based on the received signal supplied from the transmitter/receiver unit 12. The image forming unit 14 includes a tomographic image forming unit 16 and a color Doppler image forming unit 18.

The tomographic image forming unit 16, based on image capturing setting set by a user, such as a scanning range of the ultrasound beam and gain setting, for example, forms a tomographic image which is an ultrasound image, from the received signal supplied from the transmitter/receiver unit 12. In the present embodiment, the tomographic image is a B mode image representing a cross section of the target tissue as an image. The tomographic image may be a two-dimensional image or a three-dimensional image. The tomographic image is stored in a storage unit 36 and is also displayed on a display unit 32 by a display control unit 30.

The color Doppler image forming unit 18, based on the received signal obtained by Doppler measurement performed in an area set by the user, calculates distribution of the velocity of blood flow within the target issue. The color Doppler image forming unit 18 further performs conversion of the velocity to a luminance value, coloring, and the like, based on the calculated velocity distribution. Consequently, a color Doppler image having colors representing the blood flow being superposed is formed. The display control unit 30, which will be descried below, has an image synthesis function, thereby synthesizing the color Doppler image on the tomographic image formed by the tomographic image forming unit 16. Thus, a color flow mapping (CFM) image is formed.

The color Doppler image is colored with different hues and lightness in accordance with the direction and velocity of the blood flow. For example, the blood flow toward the probe 10 direction (antegrade flow) is colored with red, whereas the flow in the opposite direction (retrograde flow) is colored with blue. Dispersion (fluctuations in the flow velocity) is expressed in red or blue added with green. The flow velocity is expressed with variations of lightness and hue in accordance with the flow velocity, in such a manner that the higher the flow velocity, the higher the lightness of the color at that position. The color Doppler image is continuously updated in response to the received signal supplied from the transmitter/receiver unit 12. The color Doppler image is stored in the storage unit 36 and is displayed on the display unit 32 by the display control unit 30.

A tomographic image analysis unit 20 analyzes the tomographic image formed by the tomographic image forming unit 16 using an image processing technique, and extracts a reference portion in the target tissue. The reference portion refers to a portion in the tomographic image which exhibits a predetermined feature and is also referenced for specifying a Doppler measurement position by a Doppler measurement position specification unit 22, which will be described below. The reference portion is a contour of the heart cavity, an annulus position, and the like, when the target tissue is a heart, for example. The contour of the heart cavity is extracted by applying pattern matching and dynamic contour model in a tomographic image. Further, as the annulus position, that is, a predetermined area at the base of a valve (the mitral valve, tricuspid valve, and the like) within the heart has a high luminance in a tomographic image, a position with a luminance of a predetermined value or greater detected by luminance detection is specified as the annulus position. Active Appearance Modelling or a learning method can also be used for extracting the reference portion.

The reference portion to be extracted by the tomographic image analysis unit 20 is determined in accordance with the cross section type and the measurement item set by the user. The cross section type is “apical 4-chamber view”, for example, which is information indicating the target tissue included in a tomographic image and the cross section thereof. The measurement item is “left ventricular inflow”, for example, which is information indicating the target of the Doppler measurement. When the cross section type is an “apical 4-chamber view” and the measurement item is “left ventricular inflow blood”, the tomographic image analysis unit 20 determines that the Doppler measurement position should be specified between valves in the mitral valve, and extracts, as the reference portion, the annulus position of the mitral valve adjacent to the mitral valve.

A Doppler measurement position specification unit 22, based on the reference portion extracted by the tomographic image analysis unit 20, specifies a Doppler measurement position. For the specification, a relational expression which defines a positional relationship between a reference portion and a Doppler measurement position, for example, is used. In order to derive the relation expression, a regression analysis, which is a method for specifying the positional relationship between a reference portion and a Doppler measurement position from past data, can be used. For example, data indicating correlation between the coordinates indicating the Doppler measurement positions which were set in the past and the coordinates of the reference portions when these Doppler measurement positions are set are accumulated, and an expression indicating the relationship between the reference portion and the Doppler measurement position is derived from the data thus accumulated.

When specifying the Doppler measurement position based on the contour of the target tissue, a pattern matching method is used. Pattern data indicating correlation between a plurality of contour shape patterns of the target tissue and information of appropriate Doppler measurement positions for the respective patterns are prestored in the storage unit 36. Then, a contour shape pattern which is similar to the contour shape of the target tissue extracted by the tomographic image analysis unit 20 is specified from among the plurality of contour shape patterns, and the position correlated with this specified pattern is specified as the Doppler measurement position. It is desirable that a plurality of pattern data items are provided for each type of cross section.

As the target tissue (particularly the heart, for example) is pulsating, the positional relationship between the reference portion and the appropriate Doppler measurement position may vary depending on the time phase of the pulsation cycle. It is therefore preferable that the relation expression and the pattern data described above are provided for each time phase (in the case of the heart, early diastole, mid-diastole, end diastole, early systole, mid-systole, end systole, for example), and the relation expression and the pattern data in accordance with the time phase at the time of measurement are used. The time phase at the time of measurement may be set automatically in accordance with the measurement item, for example. A control unit 26 controls the Doppler measurement position specification unit 22, based on an organism signal measured by an organism measurement device 24 which will be described below, to specify the Doppler measurement position taking the time phase of the target tissue into consideration.

The Doppler measurement position specification unit 22 may specify the Doppler measurement position by using a color Doppler image formed by the color Doppler image forming unit 18. Here, the Doppler measurement position specification unit 22 references the distribution of velocity of blood flow within the target tissue in the color Doppler image. For measurement by continuous wave Doppler and pulsed Doppler, it is desirable to specify the Doppler measurement position at a position where the blood flow is stable. The position where the blood flow is stable refers to a position where a variation in the flow velocity is small in the distribution of the blood flow velocity and simultaneously dispersion of the velocity of the blood flow is small. In such a case, the Doppler measurement position is specified at a position in a color Doppler image where the inclination of hue is small and also an amount of green components is small in the hue, for example. Further, in order to detect a retrograde flow, it is desirable that the Doppler measurement position is specified at a position where the velocity of blood flow indicates the retrograde flow and simultaneously the velocity is the maximum. In this case, the Doppler measurement position is specified at a position in a color Doppler image where the hue is blue and the lightness thereof is the maximum.

The distribution of velocity of blood flow also varies depending on the time phase in the pulsation cycle of a target tissue. The velocity of blood flow varies in accordance with the time phase between valves of the mitral valve, for example. When the measurement item is “left ventricular inflow blood”, it is desirable that measurement is performed in a time phase and a position where the velocity of blood flow between valves in the mitral valve is the maximum. The Doppler measurement position specification unit 22 specifies the Doppler measurement position at a position where the velocity of blood flow is the maximum in a time phase when the velocity of blood flow between valves of the mitral valve is the maximum. The time phase when the velocity of blood flow between valves of the mitral valve is the maximum may be specified based on color Doppler images obtained in a plurality of time phases, or a predetermined time phase may be associated with each measurement item. As described above, when the Doppler measurement position is specified based on the blood flow distribution information, it is similarly preferable to specify the Doppler measurement position taking the time phase into consideration.

The Doppler measurement position specification unit 22 may specify the Doppler measurement position based on both the reference portion extracted by the tomographic image analysis unit 20 and the distribution of blood flow velocity. For example, a midpoint between a position specified based on the reference portion and a position specified based on the distribution of blood flow velocity may be specified as the Doppler measurement position. Alternatively, when the position specified based on the reference portion and the position specified based on the distribution of blood flow velocity are different positions, the Doppler measurement position may be specified taking the reference portion and the color Doppler information equally into consideration, such as by further specifying a Doppler measurement position based on the reference portion and the color Doppler image in another time phase.

Further, the Doppler measurement position may be specified using the reference portion and the distribution of blood flow velocity in steps. For example, the analysis range having a certain size is defined based on form information, and then the distribution of the blood flow velocity is analyzed, so that the Doppler measurement position may be specified from within the analysis range which is defined. A position in the analysis range where the blood flow velocity is the highest, for example, is specified as the Doppler measurement position.

The Doppler measurement position specification unit 22 can specify a plurality of Doppler measurement positions. For example, the annulus position of the mitral valve and the annulus position of the tricuspid valve are extracted as the reference portions, and based on these positions, the Doppler measurement positions are specified at two positions, that is, a position between valves of the mitral valve and a position between valves of the tricuspid valve. Alternatively, the Doppler measurement positions may be specified at positions where the retrograde blood flow and the antegrade blood flow are the maximum, based on the distribution of blood flow velocity.

The Doppler measurement position specification unit 22 may specify the range in which the Doppler measurement for forming a color Doppler image is performed based on the reference portion. For example, a range having a predetermined margin from the edge portion of the contour of the heart cavity is identified, and this range is determined as the range for the Doppler measurement.

The Doppler measurement position specification unit 22 may specify a tissue Doppler measurement position. The tissue Doppler measures the velocity of a predetermined portion of the target tissue. When measurement of tissue Doppler concerning the annulus portion within the heart is desired, for example, the Doppler measurement position specification unit 22 analyzes a tomographic image to extract an annulus position, and determines the annulus position as a measurement position of the tissue Doppler.

The organism signal measurement unit 24 receives an organism signal of the target tissue and generates organism signal data. The organism signal data include electrocardiographic waveforms, phonocardiography waveforms, and the like. The organism signal data are used to control the operation timing of the Doppler measurement position specification unit 22, as described above. The organism signal data are transmitted to the display control unit 30 and displayed on the display unit 32 and also stored in the storage unit 36.

A control unit 26 is a CPU, for example, and controls the whole system and also controls the operation timing of the color Doppler image forming unit 18 and the Doppler measurement position specification unit 22, using the organism signal data from the organism signal measurement unit 24. The control unit 26 also operates to perform control based on an instruction input through an input unit 34 by the user.

A Doppler waveform forming unit 28, based on a received signal obtained by Doppler measurement such as continuous wave Doppler measurement or pulsed Doppler measurement performed at a Doppler measurement position specified by the Doppler measurement position specification unit 22, generates Doppler waveforms which are the results of the waveform measurement. The Doppler waveforms, which are continuously updated, are stored in the storage unit 36 and displayed on the display unit 32 by the display control unit 30.

The display control unit 30 processes signals output from the image forming unit 14, the organism signal measurement unit 24, and the Doppler waveform forming unit 28 and outputs processed data to the display unit 32.

The display unit 32 is a monitor, such as CRT and LCD, and displays a tomographic image and a color Doppler image formed by the image forming unit 14, organism signal waveforms measured by the organism signal measurement unit 24, and Doppler waveforms formed by the Doppler waveform forming unit 28.

The input unit 34 is an interface which performs various operations of the device, and is an input device such as a keyboard, a track ball, a switch, or a dial. Further, voice input may be allowed. The input unit 34 is used for setting the type of cross section and the measurement item with respect to which the Doppler measurement is performed.

The storage unit 36 stores therein a tomographic image and a color Doppler image obtained by the image forming unit 14, a Doppler measurement position specified by the Doppler measurement position specification unit 22, organism signal waveforms measured by the organism signal measurement unit 24, and Doppler waveforms formed by the Doppler waveform forming unit 28. The storage unit 36 also stores therein programs, calculation operation systems, and estimation operation systems for actuating various functions of the ultrasonic diagnostic device. The storage unit 36 is a storage medium, such as a semiconductor memory, an optical disk, and a magnetic disk, or may be an external storage medium connected via the network.

The ultrasonic diagnostic device according to the present embodiment is configured as described above. Among the elements illustrated in FIG. 1, the transmitter/receiver unit 12, the image forming unit 14, the tomographic image analysis unit 20, the Doppler measurement position specification unit 22, the control unit 26, the Doppler waveform forming unit 28, and the display control unit 30 can be implemented using hardware such as an electronic circuit and a processor, and a device such as a memory may be used for the implementation as required. Further, the functions corresponding to the elements described above may be implemented by cooperation of hardware such as a CPU, a processor, and a memory, and software (program) which regulates the operation of the CPU and the processor. Example Doppler measurement positions specified by the ultrasonic diagnostic device according to the present embodiment will be described hereinafter.

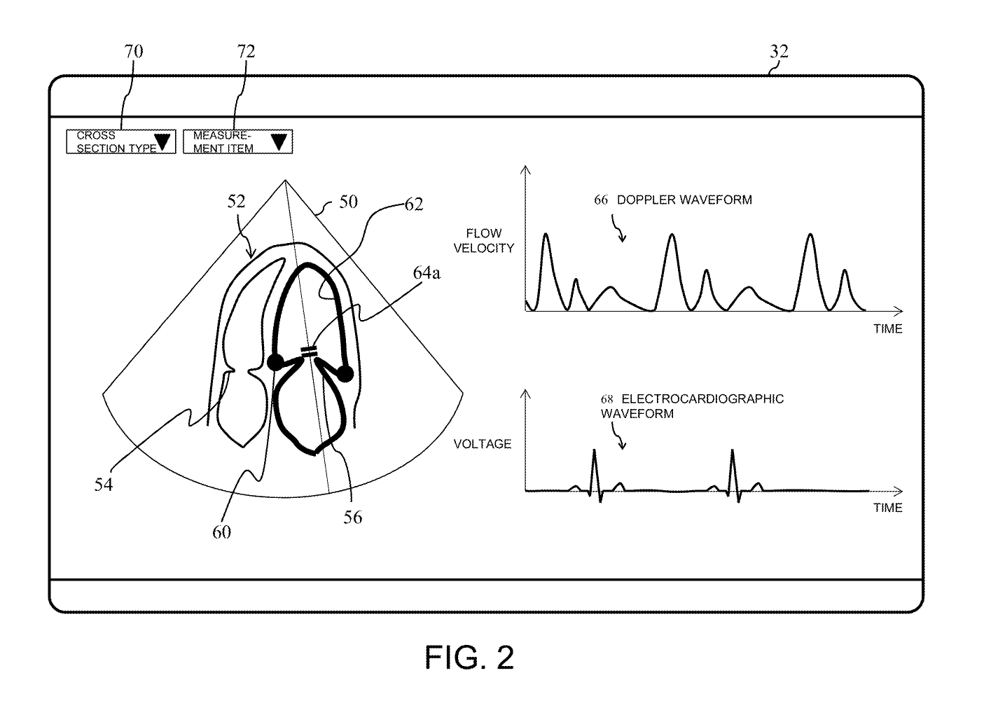

FIG. 2 is a diagram illustrating an example Doppler measurement position specified based on the reference portion. FIG. 2 will be described with reference to FIG. 1. FIG. 2 shows a screen displayed on the display unit 32, and includes a B-mode image 50 formed by the tomographic image forming unit 16 on the left side, a Doppler waveform 66 formed by the Doppler waveform forming unit 28 and an electrocardiographic waveform 68 measured by the organism signal measurement unit 24 on the right side.

The B-mode image 50 is a tomographic image of the heart 52, which is a target tissue, and illustrates cross sections of the left ventricle, the left atrium, the right ventricle, and the right atrium of the heart. The heart 52 includes the tricuspid valve 54 located between the right ventricle and the right atrium and the mitral valve 56 located between the left ventricle and the left atrium, and these valves are also shown in the B-mode image 50.

The annulus position 60 is a portion located at the root of the mitral valve 56, and is a position which is specified by executing luminance detection with respect to the B-mode image 50 by the tomographic image analysis unit 20. FIG. 2 illustrates an example in which the measurement item is set to “left ventricular inflow blood” and the Doppler measurement position 64a is specified based on the annulus position 60 by the Doppler measurement position specification unit 22. The annulus position 60 serving as a reference for specifying the Doppler measurement position 64a is displayed in an emphasized manner to allow a user to recognize the portion with reference to which the Doppler measurement position 64a is specified. The annulus position 60 may or may not be emphasized. Further, the Doppler measurement position 64a may be specified based on the contour of the heart cavity 62 or on both the annulus position 60 and the heart cavity contour 62.

It is difficult to directly set a position whose shape cannot be represented on the B-mode image 50, such as a Doppler measurement position of blood flow, automatically. Therefore, in the present embodiment, a position to be subjected to the Doppler measurement is specified from the reference portion within the heart. Setting the Doppler measurement position to a position with reference to the heart cavity, for example, enables setting of the Doppler measurement position with high accuracy in a stable manner. Further, when the Doppler measurement in a valve portion is desired, it is possible to set the Doppler measurement position based on a position which is close to a desired Doppler measurement position and which is also an annulus position with a relatively small variation in position fluctuation.

A cursor indicating the Doppler measurement position is displayed on the Doppler measurement position 64a, so that the user can identify the Doppler measurement position which is specified. A cursor indicates a sample gate corresponding to a gate for sampling the received signals in a pulsed Doppler mode. In continuous wave Doppler mode, a cursor indicates a sample volume which is a cross point between the transmitting beam and the received beam. In FIG. 2, the cursor which is shown is a cursor in the pulsed Doppler mode.

The Doppler waveform 66 is a waveform indicating a result of the Doppler measurement in the Doppler measurement position 64a indicated by the cursor. In the Doppler wave form, the horizontal axis indicates time, and the vertical axis indicates a velocity of blood flow. The electrocardiographic waveform 68 is a waveform which electrically indicates the movements of the heart 52, and is generated based on an organism signal obtained by the organism signal measurement unit 24. In the electrocardiographic waveform 68, the horizontal axis indicates time and the vertical axis indicates a voltage. The electrocardiographic waveform 68 enables the user to understand the relationship between the Doppler waveform 66 and the time phase in the pulsation cycle of the heart 52.

A cross section type box 70 indicates a type of the cross section of the B-mode image 50. The cross section type may be input by the user through the input unit 34. Alternatively, the cross section type may be automatically determined by performing image processing of the B-mode image by the tomographic image analysis unit 20. The cross section type of the B-mode image 50 illustrated in FIG. 2 is an “apical 4-chamber view”. A measurement item box 72 indicates a subject of the Doppler measurement. The measurement item is input by the user through the input unit 34. Based on the measurement item, which portion is to be extracted by the tomographic image analysis unit 20 as a reference portion, or to which position the Doppler measurement position is to be specified with reference to the reference portion, is determined.

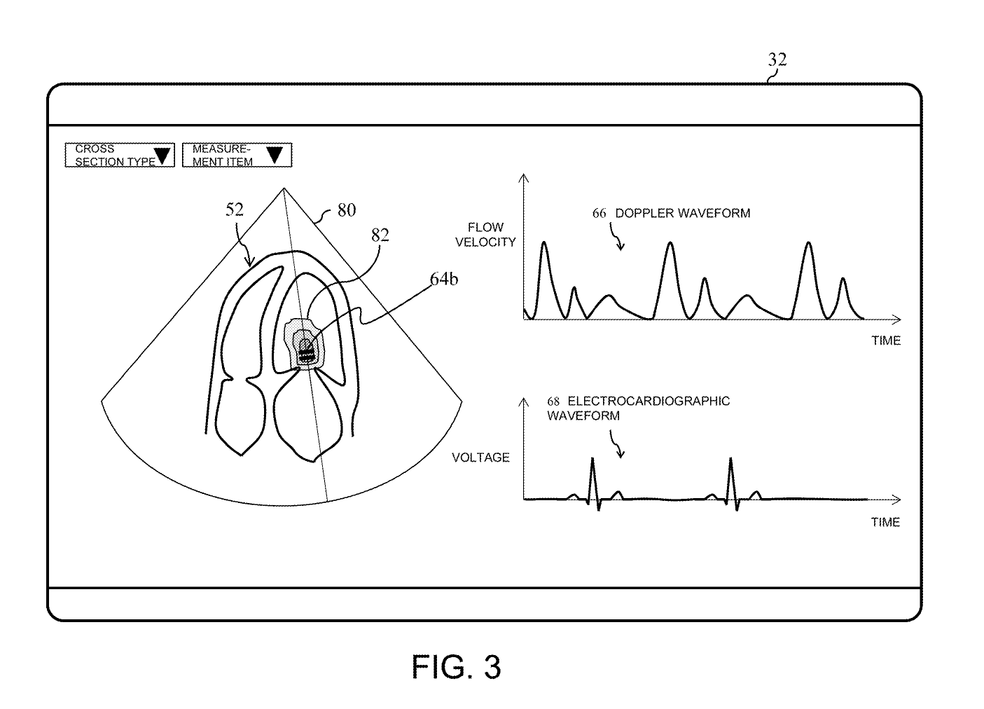

FIG. 3 illustrates an example Doppler measurement position specified based on the distribution of the blood flow velocity. FIG. 3 illustrates a CFM image 80 which shows the distribution of blood flow velocity 82. In the flow velocity distribution 82, the antegrade flow is colored with red and the retrograde flow is colored with blue, and the flow velocity is represented by lightness. In the left ventricle diastolic phase (left ventricular inflow phase) illustrated in FIG. 3, the flow velocity distribution 82 is represented on the inner cavity side of the left ventricle. As the flow velocity distribution 82 is represented in a jet pattern from the leaflet toward the inner cavity side of the left ventricle, it is desirable that the Doppler measurement is performed in a portion within the jet pattern where the flow velocity is stable. Therefore, a position in the data of the flow velocity distribution 82 where the flow velocity is high (the lightness is high in the flow velocity distribution 82, for example) and the dispersion of the flow velocity is small (the amount of the green component of hue is low in the flow velocity distribution 82, for example) is detected. This detected position is determined as a detailed estimated Doppler measurement position.

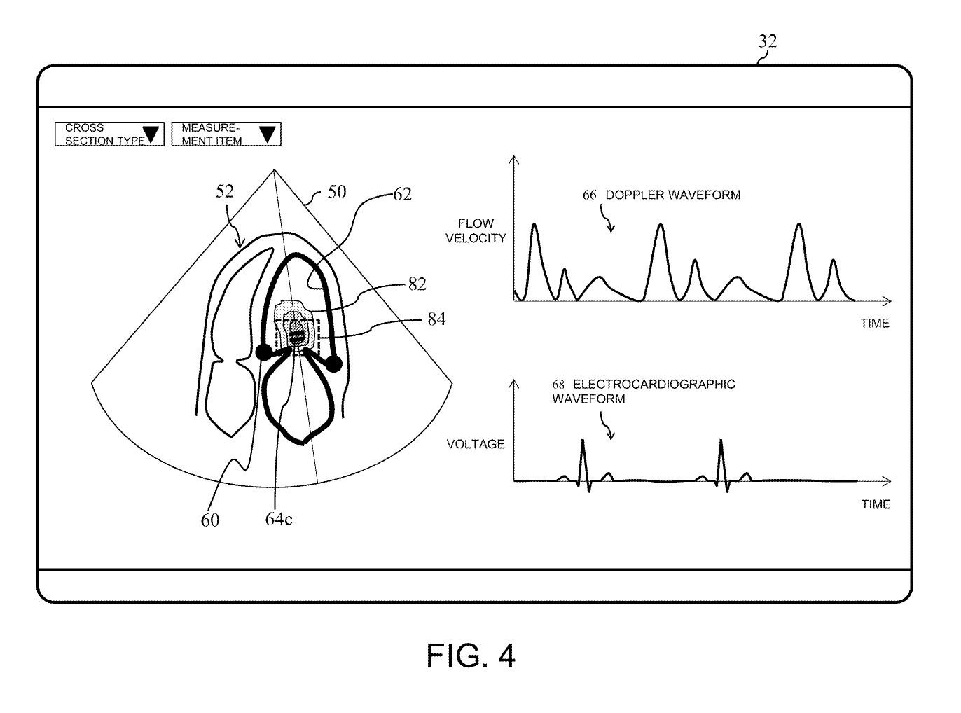

FIG. 4 illustrates an example Doppler measurement position specified based on the reference portion and the velocity distribution of blood flow. An analysis range 84 is first defined based on the annulus position 60 or the heart cavity contour 62. The analysis range 84 is a range of positions that can be an appropriate Doppler measurement position. The measurement item may be taken into consideration in defining the analysis range 84. While in FIG. 4 the analysis range 84 is of a rectangular shape, the analysis range 84 may be a circular shape or an elliptical shape, for example, or may be discrete ranges. Subsequently, based on the velocity distribution 82 of blood flow, the Doppler measurement position 64c is specified from within the analysis range 84 that is defined. For example, a position within the analysis range 84 where the blood flow velocity is the maximum is specified as the Doppler measurement position 64c.

Use of both the reference portion and the blood flow velocity distribution for specifying the Doppler measurement position as in the example illustrated in FIG. 4 can increase the accuracy of the specification. In a case where the Doppler measurement position is specified based only on the reference portion, for example, as statistical methods including regression analysis, pattern matching, and so on, are used to specify the Doppler measurement position, there is a possibility that a slight difference will exist between the Doppler measurement position which is specified and the correct Doppler measurement position (e.g. a position where the blood flow velocity is the maximum). When the Doppler measurement position is specified based only on the blood flow velocity distribution, on the other hand, in a case where a position where the blood flow velocity is the maximum (between valves of the tricuspid valve, for example) is present on the flow velocity distribution 82 in addition to a position desired by the user (between valves of the mitral valve, for example), the Doppler measurement position may be specified between valves of the tricuspid valve in spite of the user's intention. However, defining the analysis range 84 based on the reference portion and the measurement item can prevent specification of the Doppler measurement position which is not desired by the user, and consideration of the blood flow velocity distribution within the analysis range 84 enables specification of the Doppler measurement position to an appropriate position for each measurement.

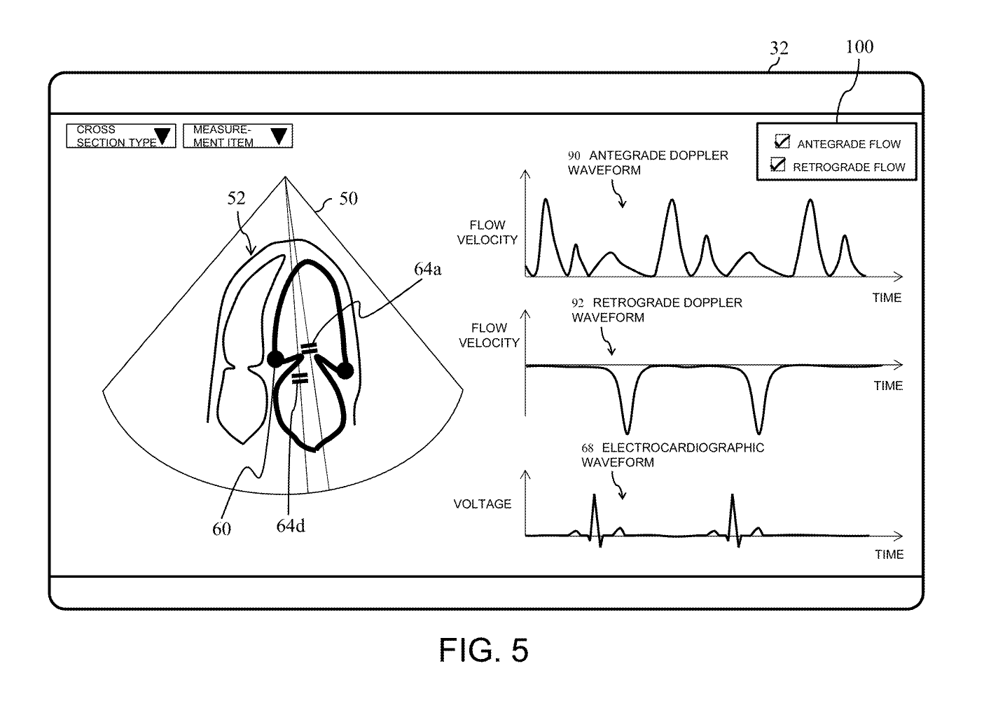

FIG. 5 illustrates an example in which a plurality of Doppler measurement positions are specified based on the reference portion. It is possible to specify a plurality of Doppler measurement positions. When the measurement item is set to “antegrade flow and retrograde flow of the mitral valve”, for example, a Doppler measurement position 64a corresponding to the antegrade flow of the mitral valve is first specified based on the annulus position 60 or the heart cavity contour 62 as in the example illustrated in FIG. 2. Then, based on the annulus position 60 or the heart cavity contour 62, a Doppler measurement position 64d is specified at a position where the retrograde flow of the mitral valve the mitral regurgitation occurs. Of course, the Doppler measurement positions 64a and 64d are specified using different relational expressions or patterns. The Doppler measurement positions 64a and 64d may be specified based on the distribution of blood flow velocity as illustrated in FIG. 3, or specified based on both the reference portion and the distribution of velocity of blood flow as illustrated in FIG. 4. Further, a plurality of Doppler measurement positions may be specified for different valves, such as the left ventricular inflow blood (the mitral valve) and the right ventricular inflow blood (the tricuspid valve), rather than the antegrade and retrograde flows for a single valve.

The antegrade flow Doppler waveform 90 is a waveform indicating the results of the Doppler measurement at the Doppler measurement position 64a, and the retrograde flow Doppler waveform 92 is a waveform indicating the results of the Doppler measurement at the Doppler measurement position 64d. The two waveforms can be displayed simultaneously. Further, the user can click a check box 100 to make the antegrade flow Doppler waveform 90 or the retrograde flow Doppler waveform 92 disappear. It is preferable that, at this time, the cursor indicating the Doppler measurement position corresponding to the Doppler waveform which is not displayed is shown in a dashed line or in a different color. Also, the measurement period may be limited to the systole phase in the cardiac pulsation cycle based on the organism signal. There may be no retrograde flow for some examinees, in which case, information showing “no retrograde flow”, in place of the retrograde flow Doppler waveform 92, may be displayed on the screen.

Simultaneous display of a plurality of cursors indicating a plurality of Doppler measurement positions enables the user to simultaneously identify the plurality of Doppler measurement positions which are specified. It is also possible to change the display of Doppler waveforms corresponding to a plurality of Doppler measurement positions with a simple operation. Further, display of the Doppler waveform of the antegrade flow and the Doppler waveform of the retrograde flow along with the electrocardiographic waveforms in the same time phase enables the user to easily understand the correlation of the antegrade flow Doppler waveform 90 and the retrograde flow Doppler waveform 92 with the electrocardiographic waveform 68.

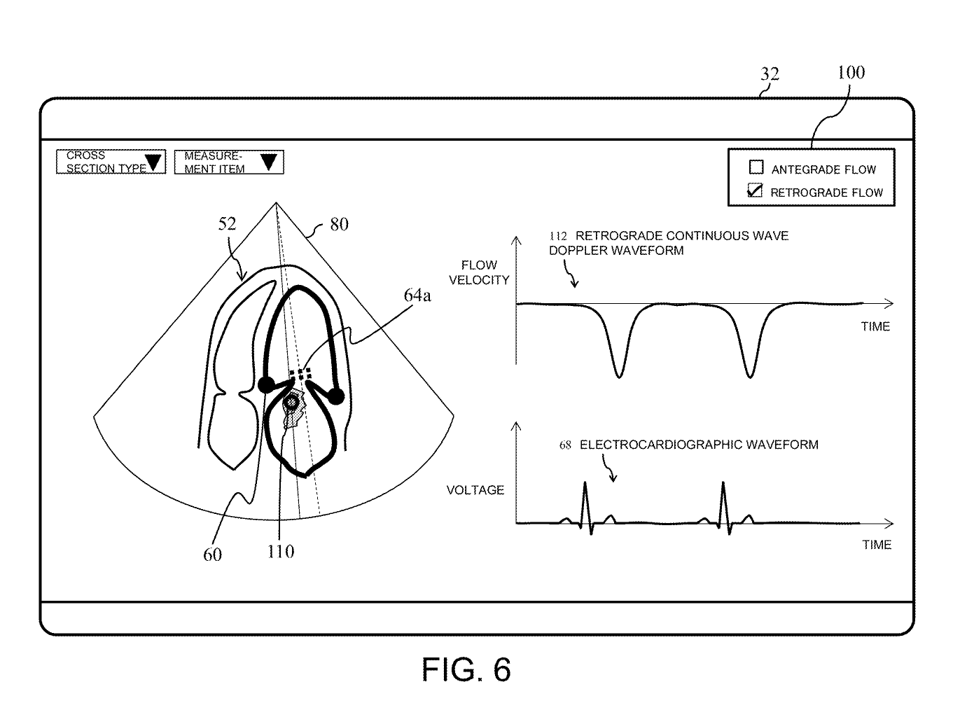

FIG. 6 illustrates a state in which the Doppler measurement in a retrograde flow position is changed from the pulsed Doppler mode to the continuous wave Doppler mode. As it is generally likely that the blood flow velocity will be high in a retrograde flow position, in a retrograde flow position, measurement is performed preferably in the continuous wave Doppler mode which is suitable for measurement of flow at high velocities. In the example in FIG. 6, when the measurement item is “the mitral regurgitation”, for example, and the Doppler measurement position 100 for measuring the retrograde flow is specified, the mode of the Doppler measurement in this Doppler measurement position is automatically changed from the pulsed Doppler mode to the continuous wave Doppler mode. Further, a retrograde continuous wave Doppler waveform 112 measured by the continuous wave Doppler mode is then displayed. It is preferable that the shape of the cursor in the continuous wave Doppler mode is different from that of the cursor in the pulsed Doppler mode. In the example illustrated in FIG. 6, the cursor indicating the Doppler measurement position 110 in the continuous wave Doppler mode is of a circular shape. Also, in consideration of the velocity of the blood flow, only when the velocity of the blood flow in a retrograde flow position is equal to a predetermined value or greater, the Doppler mode may be changed to the continuous wave Doppler mode. Automatic change of the Doppler mode in a retrograde flow position to the continuous wave Doppler enables selection of an appropriate Doppler mode while eliminating time and labor for the user' s operation.

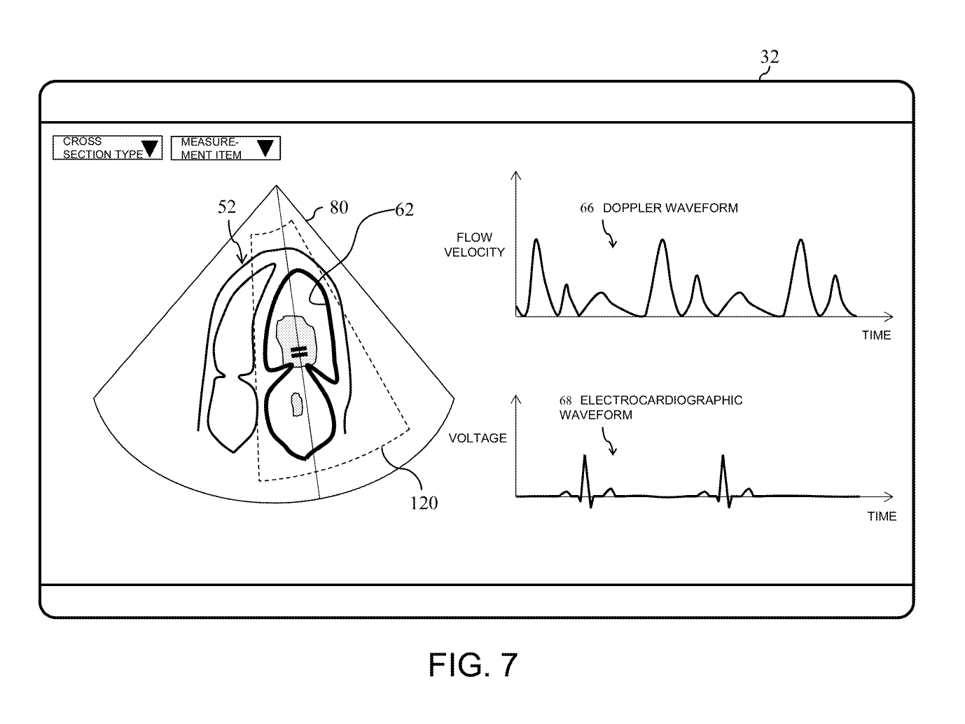

FIG. 7 is a diagram illustrating an example color Doppler measurement range specified based on the reference portion. In the example illustrated in FIG. 7, the cross section type is “apical 4-chamber view” and the measurement item is “left ventricular inflow blood”, with a color Doppler measurement range 120 being defined so as to enclose the whole left ventricle. As the color Doppler measures the blood flow in a predetermined range, the color Doppler measurement range 120 is defined based on the heart cavity contour 62 which is a contour of the left ventricle. Specifically, a range with a predetermined margin from the edge of the heart cavity contour 62 is set as the color Doppler measurement range 120. Alternatively, a sector portion including the left and right annulus positions 60 may be set as the color Doppler measurement range 120. Automatic specification of the color Doppler measurement range enables setting of an appropriate color Doppler measurement range and also can eliminate the user's labor.

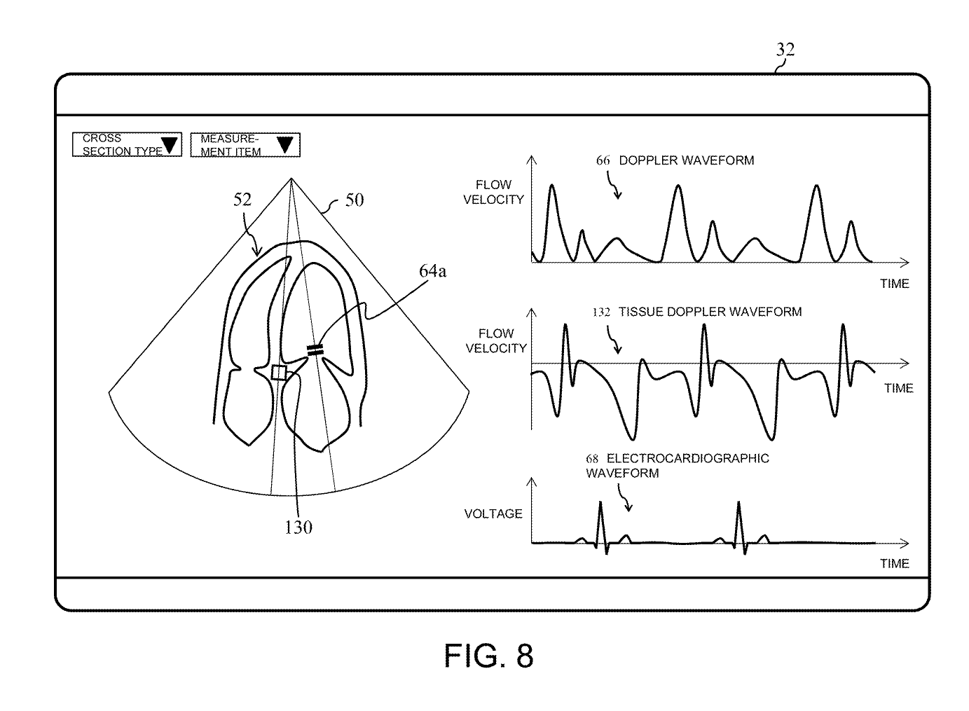

FIG. 8 is a diagram illustrating an example measurement position of tissue Doppler specified based on the reference portion. In the example of FIG. 8, the measurement item is set to “left ventricular inflow blood and the mitral valve annulus velocity”. While in this example the Doppler measurement position 64a for the left ventricular inflow blood is specified in a manner similar to that in the example illustrated in FIG. 2, in the example illustrated in FIG. 8, a Doppler measurement position 130 in the tissue Doppler mode for measuring the mitral valve annulus velocity is automatically specified. The Doppler measurement position 130 is specified based on the reference portion. For example, similar to the example of FIG. 2 and the like, an annulus position is extracted from the tomographic image, and the extracted annulus position is specified as the Doppler measurement position 130. As illustrated in FIG. 8, the Doppler waveform 66 of the left ventricular inflow blood, the tissue Doppler waveform 132 which is a result of the Doppler measurement at the Doppler measurement position 130, and the electrocardiographic waveform 68, are displayed in parallel. Automatic specification of the tissue Doppler measurement position enables specification of the tissue Doppler measurement position to an appropriate position and also can eliminates the user's labor.

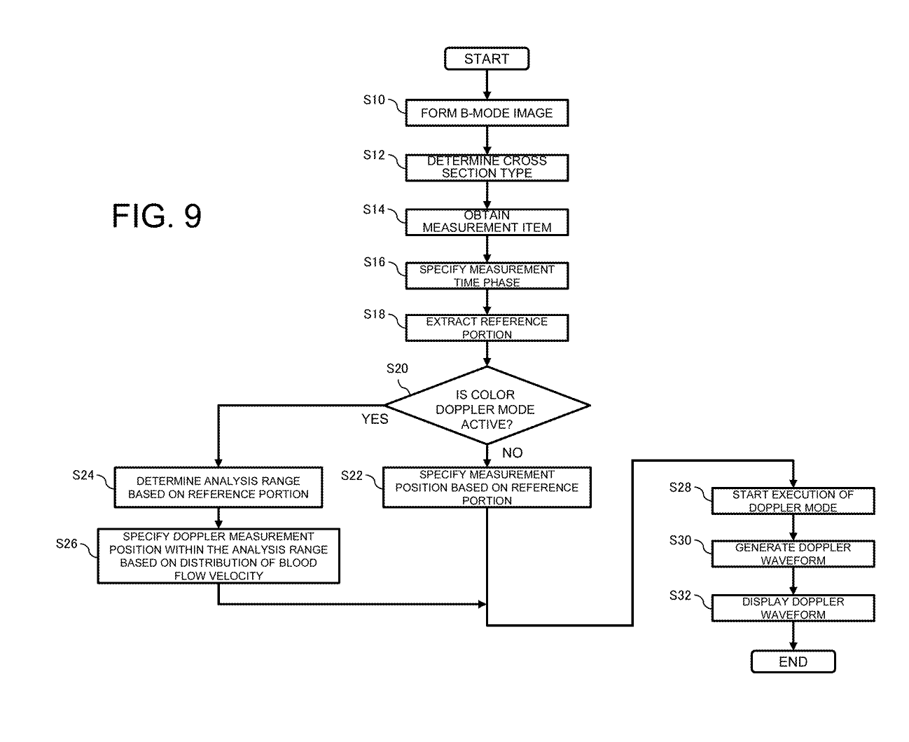

The flow of processing of the ultrasonic diagnostic device according to the present embodiment will be described below. FIG. 9 is a flowchart showing a flow of the operation of the ultrasonic diagnostic device according to the present embodiment. The flowchart in FIG. 9 will be described with reference to FIG. 1.

In step S10, the tomographic image forming unit 16 forms a B-mode image which is a tomographic image, based on a signal from the transmitter/receiver unit 12.

In step S12, the tomographic image analysis unit 20 determines a cross section type of the B-mode image formed in step S10, using an image recognition technique. As the image recognition technique, known techniques such as a pattern matching method, a subspace method, a Bag of Features method, for example, can be used. The cross section types include, in the case of the heart, an apical 2-chamber view, an apical 3-chamber view, an apical 4-chamber view, a parasternal long-axis cross section, a parasternal short-axis cross section, and the like.

In step S14, the Doppler measurement position specification unit 22 obtains a measurement item set by the user. The measurement item may be, for example, the left ventricular inflow, the mitral regurgitation, and the like.

In step S16, based on the measurement item obtained in step S14, the time phase which is the most suitable for measuring the measurement target indicated by that measurement item is specified. When the measurement item is “the mitral valve regurgitation”, the time phase is specified to the systole phase.

In step S18, the tomographic image analysis unit 20, based on the measurement item obtained in step S14, extracts a reference portion for specifying the Doppler measurement position from the B-mode image at the time phase specified in step S16.

In step S20, the control unit 26 determines whether or not the color Doppler mode is active.

If it is determined in step S20 that the color Doppler mode is not active, the Doppler measurement position specification unit 22 specifies, in step S22, a Doppler measurement position based on the reference portion extracted in step S18.

If it is determined in step S20 that the color Doppler mode is active, the Doppler measurement position specification unit 22 defines, in step S24, an analysis range of a candidate Doppler measurement position based on the reference portion extracted in step S18.

In step S26, the Doppler measurement position specification unit 22 specifies, as the Doppler measurement position, a position within the analysis range defined in step S24 where the velocity of blood flow is the highest based on the distribution of the blood flow velocity.

Upon specification of the Doppler measurement position in step S22 or S26, the Doppler waveform forming unit 28 automatically starts the Doppler measurement in step S28. Prior to the start of the Doppler measurement, the B-mode image is frozen on the display unit 32.

In step S30, the Doppler waveform forming unit 28 performs the Doppler measurement and generates a Doppler waveform in a Doppler measurement position specified in step S22 or S26.

In step S32, the display control unit 30 causes the display unit 32 to display the Doppler waveform generated in step S30. In addition to the Doppler waveform, a B-mode image or a color Doppler image, and a cursor indicating the Doppler measurement position shown on these images are displayed, and also an electrocardiographic waveform measured by the organism signal measurement device 24 is displayed in parallel to the Doppler waveform.

As described above, according to the present embodiment, specification of the Doppler measurement position based on a reference portion in a target tissue specified on the tomographic image enables automatic setting of the Doppler measurement position with high accuracy. Further, specification of the Doppler measurement position in further consideration of the distribution of blood flow velocity increases the accuracy of the Doppler measurement position.

### REFERENCE SIGN LIST

10 probe, 12 transmitter/receiver unit, 14 image forming unit, 16 tomographic image forming unit, 18 color Doppler image forming unit, 20 tomographic image analysis unit, 22 Doppler measurement position specification unit, 24 organism signal measurement device, 26 control unit, 28 Doppler waveform forming unit, 30 display control unit, 32 display unit, 34 input unit, 36 storage unit, 50 B-mode image, 52 heart, 54 tricuspid valve, 56 the mitral valve, 60 annulus position, 62 heart cavity contour, 64a to 64d Doppler measurement position, 66 Doppler waveform, 68 electrocardiographic waveform, 80 CFM image, 82 flow velocity distribution, 84 analysis range, 90 antegrade flow Doppler waveform, 92 retrograde flow Doppler waveform, 100 check box, 110 Doppler measurement position in waveform Doppler mode, 112 retrograde flow Doppler waveform, 120 color Doppler measurement range, 130 Doppler measurement position in tissue Doppler mode, 132 tissue Doppler waveform.

1. An ultrasonic diagnostic device, comprising:
a tomographic image forming unit configured to form, based on a received signal obtained by transmitting and receiving an ultrasound wave to and from a beam scanning area including a target tissue in which blood flows, a tomographic image of the target tissue;
a tomographic image analysis unit configured to analyze the tomographic image to extract a reference portion in the target tissue;
a position specification unit configured to specify a Doppler measurement position used for measuring motion of a blood flow in a position within the target tissue which is different from the reference portion, based on the reference portion; and
a Doppler waveform forming unit configured to form a Doppler waveform showing a movement of blood flow in the Doppler measurement position, based on a received signal obtained by transmitting and receiving an ultrasound wave to the Doppler measurement position.

2. The ultrasonic diagnostic device according to claim 1, further comprising:
a blood flow information generation unit configured to generate blood flow information indicating a spatial distribution of velocity of a blood flow within the target tissue, based on the received signal,
wherein the position specification unit specifies the Doppler measurement position based on the reference portion and the blood flow information.

3. The ultrasonic diagnostic device according to claim 2, wherein
the tomographic image analysis unit defines an analysis range based on the reference portion, and
the position specification unit specifies the Doppler measurement position based on a portion in a spatial distribution of the velocity of the blood flow corresponding to a distribution of the velocity within the analysis range.

4. The ultrasonic diagnostic device according to claim 3, wherein the position specification unit specifies the Doppler measurement position based on a position within the analysis range where the velocity of blood flow is the maximum.

5. The ultrasonic diagnostic device according to claim 2, wherein
the position specification unit specifies the Doppler measurement position based on the reference portion and the blood flow information in a specific time selected in a pulsation cycle of the target tissue.

6. The ultrasonic diagnostic device according to claim 5, wherein
the specific time is a time phase in which a blood flow in a specific direction is expressed in the Doppler measurement position.

7. The ultrasonic diagnostic device according to claim 1, wherein
the target tissue is a heart, and
the reference portion is an annulus portion of the heart.

8. The ultrasonic diagnostic device according to claim 1, wherein
the target tissue is a heart, and
the reference portion is a contour of a heart cavity.

9. The ultrasonic diagnostic device according to claim 1, wherein
the position specification unit specifies a plurality of Doppler measurement positions within the target tissue based on the reference portion.

10. The ultrasonic diagnostic device according to claim 2, wherein
the position specification unit specifies a plurality Doppler measurement positions within the target tissue in accordance with a plurality of flow directions of blood flow based on the reference portion.

11. The ultrasonic diagnostic device according to claim 9, further comprising:
a measurement position selection unit configured to select from among the plurality of Doppler measurement positions a specific Doppler measurement position concerning which a Doppler waveform is to be displayed.

12. The ultrasonic diagnostic device according to claim 10, further comprising:
a measurement position selection unit configured to select from among the plurality of Doppler measurement positions a specific Doppler measurement position concerning which a Doppler waveform is to be displayed.
